(19) United States
(12) Patent Application Publication
(10) Pub. No.: US 2018/0214670 A1
(43) Pub. Date: Aug. 2, 2018

(54) DIAGNOSTIC IMAGING CATHETER

(71) Applicant: TERUMO KABUSHIKI KAISHA, Tokyo (JP)

(72) Inventors: Yuuki Sakaguchi, Fujinomiya-shi (JP); Masanori ATO, Bear City, DE (US)

(73) Assignee: TERUMO KABUSHIKI KAISHA, Tokyo (JP)

(21) Appl. No.: 15/884,771

(22) Filed: Jan. 31, 2018

(30) Foreign Application Priority Data
Jan. 31, 2017 (JP) 2017-015608

(51) Int. Cl. A61M 25/01; A61B 8/12; A61B 5/00

(52) U.S. Cl. CPC A61M 25/0144; A61B 5/0084; A61B 5/0066; A61B 8/12

(57) ABSTRACT

A diagnostic imaging catheter has flexibility at a distal end and has relatively high rigidity at a proximal end while avoiding an increase in outer diameter of a sheath. The diagnostic imaging catheter includes a rotatable drive shaft having a distal portion provided with an ultrasound transducer, a sheath in which the drive shaft is positioned and which extends in an axial direction, and a rigidity changing part provided inside the sheath and configured to make the rigidity of the drive shaft higher at the proximal end than at the distal end.

### CROSS-REFERENCES TO RELATED APPLICATIONS

This application is based on and claims priority to Japanese Application No. 2017-015608 filed on Jan. 31, 2017, the entire content of which is incorporated herein by reference.

### TECHNICAL FIELD

The present invention generally relates to a catheter, more particularly a diagnostic imaging catheter.

### BACKGROUND DISCUSSION

Heretofore, medical devices that are used to acquire a diagnostic image available for diagnosing, for example a diagnostic image of a diseased site in the living body, include a diagnostic imaging catheter that is used for diagnostic imaging apparatuses for use in intravascular ultrasound (IVUS) or optical coherence tomography (OCT).

The diagnostic imaging catheter includes a rotatable drive shaft having a distal end provided with a signal transmitting and receiving unit, and a sheath into which the drive shaft is inserted. During use of the diagnostic imaging catheter, what is called a pull-back operation (pull-back) which moves the drive shaft from the distal side to the proximal side by moving the drive shaft backward while rotating the drive shaft and a push-in operation which pushes the drive shaft into the distal side are performed. An example is disclosed in Japanese Application Publication No 2015-119994.

### SUMMARY

Such a diagnostic imaging catheter is required to have, at the distal side (distal end), the flexibility following a guide wire from a viewpoint of operability, and to have relatively high rigidity at the proximal side (proximal end) to improve pushability (transmissibility of push-in force). In order to improve the rigidity of the proximal portion, for example, the wall thickness of the sheath may be increased with the inner diameter of the sheath constant, but increasing the outer diameter of the sheath is not desirable from a viewpoint of compatibility with respect to other devices. That is, increasing the outer diameter of the sheath reduces the choices of other devices that can be inserted into an introducer sheath at the same time with the diagnostic imaging catheter.

The diagnostic imaging catheter disclosed here exhibits flexibility at a distal side (distal end) and has relatively high rigidity at a proximal side (proximal end) while avoiding an increase in outer diameter of the sheath.

An example of the diagnostic imaging catheter disclosed here includes a rotatable drive shaft having a distal end provided with a signal transmitting and receiving unit, a sheath into which the drive shaft is inserted and which extends in an axial direction, and a rigidity changing part provided inside the sheath and configured to make rigidity of the drive shaft higher at a proximal side than at a distal side.

According to the diagnostic imaging catheter configured as described above, the rigidity changing part is able to make the rigidity of the drive shaft higher (greater) at the proximal side than at the distal side. Moreover, the rigidity changing part is located inside the sheath and is, therefore, able to prevent an increase in outer diameter of the sheath. Accordingly, a diagnostic imaging catheter which has flexibility at a distal side and has high rigidity at a proximal side while preventing an increase in outer diameter of the sheath can be provided.

According to another aspect, a diagnostic imaging catheter comprises an axially extending sheath that includes a lumen extending along an axial extent of the sheath, and an axially extending rotatable drive shaft positioned in the lumen of the sheath, with the drive shaft being axially movable and possessing a distal end at which is located a signal transmitting and receiving unit that transmits signals towards an inner surface of a lumen in a living body and receives reflected signals that are reflected from the inner surface of the lumen in the living body. The drive shaft possesses a distal portion terminating in a distal direction at the distal end of the drive shaft, and the drive shaft also possesses a proximal portion terminating in a proximal direction at a proximal end of the drive shaft. A rigidity changing part is positioned in the lumen in the sheath and imparts a higher rigidity to a proximal portion of the drive shaft than the distal portion of the drive shaft. The rigidity changing part is connected to the drive shaft so that axial movement of the drive shaft results in axial movement of the rigidity changing part, with the rigidity changing part possessing a distal portion that terminates in a distal direction at the distal end of the rigidity changing part. The distal end of the rigidity changing part is axially spaced in the proximal direction from the distal end of the drive shaft so that the distal portion of the drive shaft does not axially overlap the rigidity changing part. The rigidity changing part axially overlaps the drive shaft.

### BRIEF DESCRIPTION OF THE DRAWINGS

FIG. 1 is a plan view illustrating a state in which an external apparatus is connected to a diagnostic imaging catheter according to a first embodiment representing one example of the diagnostic imaging catheter disclosed here.

FIGS. 2(A) and 2(B) are plan views schematically illustrating an overall configuration of the diagnostic imaging catheter according to the first embodiment, in which FIG. 2(A) is a diagram illustrating a state obtained before the pull-back operation is performed and FIG. 2(B) is a diagram illustrating a state obtained when the pull-back operation is being performed.

FIG. 3 is an enlarged cross-sectional view illustrating a configuration of a distal side of the diagnostic imaging catheter according to the first embodiment.

FIG. 4(A) is a cross-sectional view taken along section line 4A-4A in FIG. 3, FIG. 4(B) is a cross-sectional view taken along section line 4B-4B in FIG. 3, and FIG. 4(C) is a cross-sectional view taken along section line 4C-4C in FIG. 3.

FIG. 5 is an enlarged cross-sectional view illustrating a configuration of a proximal side of the diagnostic imaging catheter according to the first embodiment.

FIG. 6 is an enlarged cross-sectional view illustrating a configuration of a distal side of a diagnostic imaging catheter according to a modification example of the first embodiment.

FIG. 7(A) is a cross-sectional view taken along section line 7A-7A in FIG. 6, FIG. 7(B) is a cross-sectional view taken along section line 7B-7B in FIG. 6, and FIG. 7(C) is a cross-sectional view taken along section line 7C-7C in FIG. 6.

FIG. 8 is an enlarged cross-sectional view illustrating a configuration of a distal side of a diagnostic imaging catheter according to a second embodiment.

FIG. 9 is an enlarged cross-sectional view illustrating a configuration of a distal side of a diagnostic imaging catheter according to a third embodiment.

FIG. 10 is a diagram used to explain a modification example of electric signal cables.

FIG. 11 is a diagram used to explain a modification example of a diagnostic imaging catheter.

### DETAILED DESCRIPTION

### First Embodiment

Hereinafter, embodiments of a diagnostic imaging catheter representing examples of the inventive diagnostic imaging catheter disclosed here will be described with reference to the accompanying drawings. Furthermore, the following description should not be construed to limit the technical scope set forth in the claims or the meanings of terms. Moreover, dimensional ratios illustrated in the drawings are exaggerated for the purpose of illustration and may be different from the actual ratios.

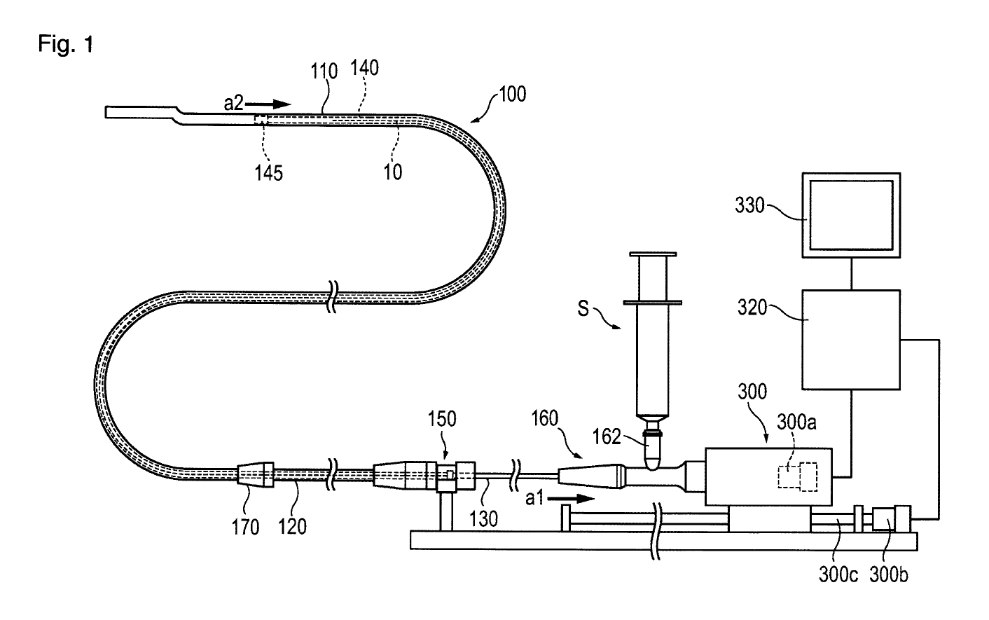

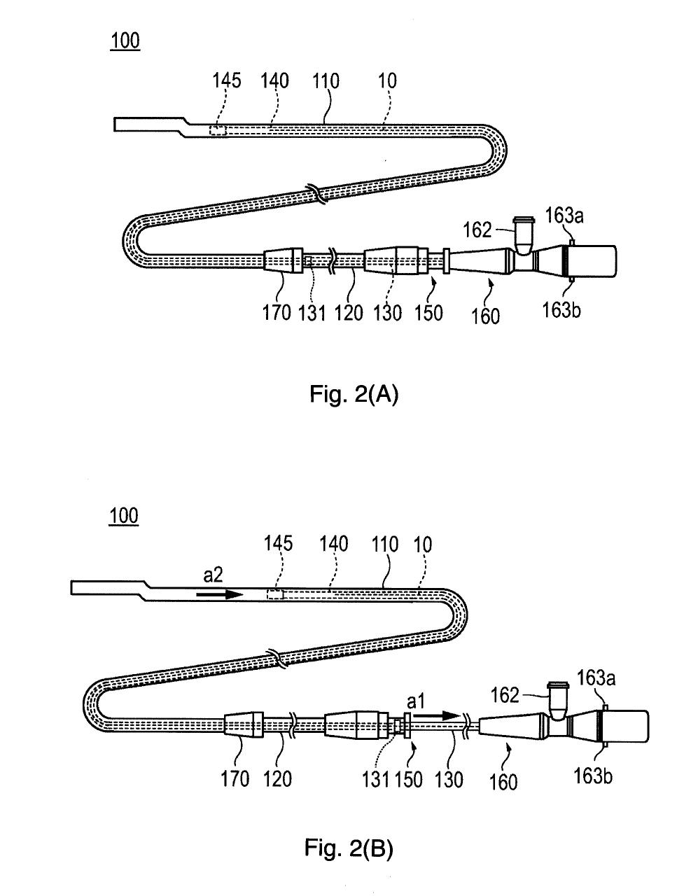

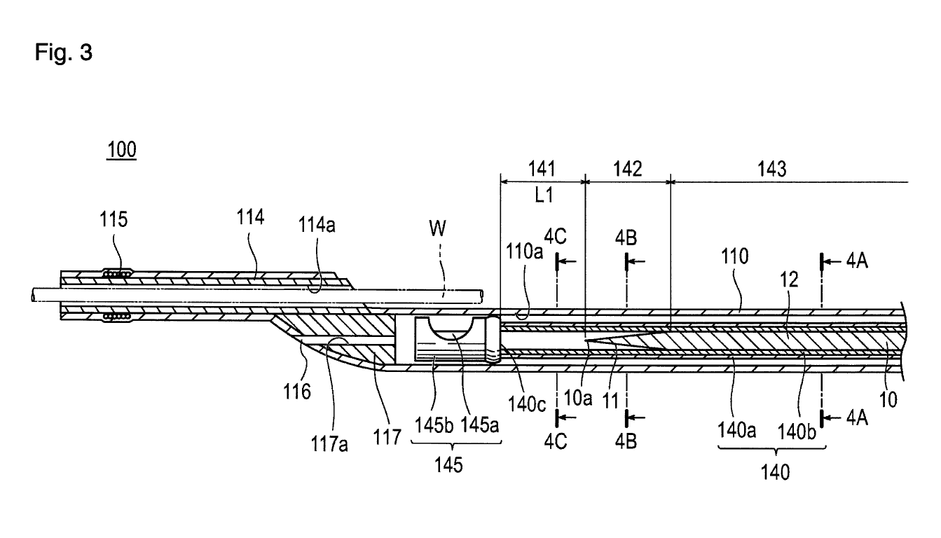

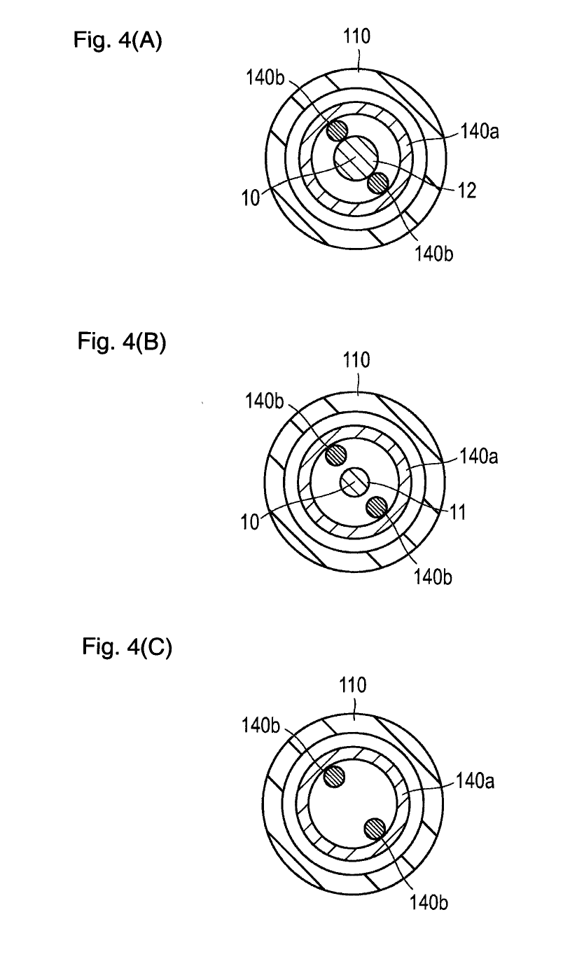

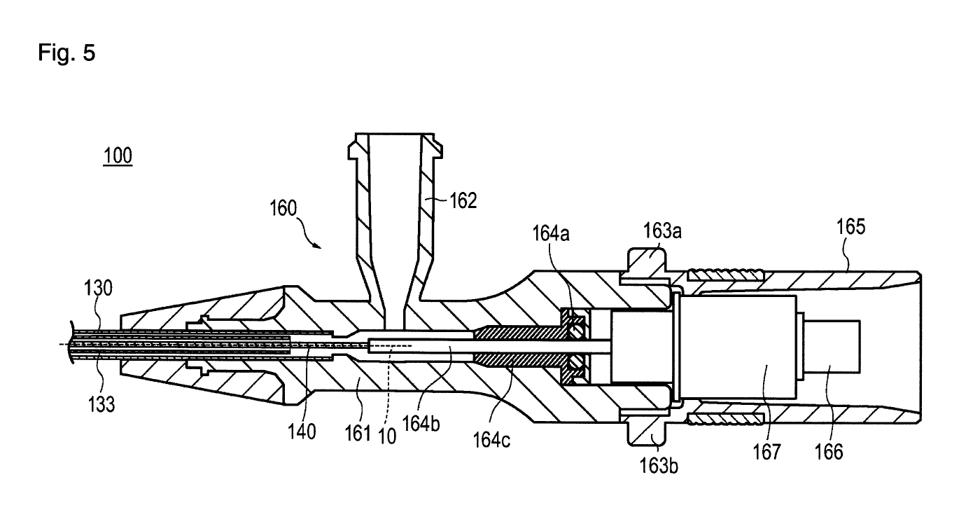

FIG. 1 is a plan view illustrating a state in which an external apparatus 300 is connected to a diagnostic imaging catheter 100 according to a first embodiment, FIGS. 2(A) and 2(B) are diagrams schematically illustrating an overall configuration of the diagnostic imaging catheter 100 according to the first embodiment, FIG. 3 and FIGS. 4(A), 4(B), and 4(C) are diagrams illustrating a configuration of a distal side of the diagnostic imaging catheter 100 according to the first embodiment, and FIG. 5 is a diagram illustrating a configuration of a proximal side of the diagnostic imaging catheter 100 according to the first embodiment.

The diagnostic imaging catheter 100 according to the first embodiment is applied to intravascular ultrasound (IVUS). As illustrated in FIG. 1, the diagnostic imaging catheter 100 is driven by being connected to the external apparatus 300. Hereinafter, the diagnostic imaging catheter 100 is described with reference to FIG. 1 to FIG. 5.

As illustrated in FIG. 1 and FIGS. 2(A) and 2(B), the diagnostic imaging catheter 100 includes a sheath 110, which is inserted into the body lumen in a living body, an outer tube 120, which is provided at the proximal side or proximal end of the sheath 110, an inner shaft 130, which is inserted into or positioned in the outer tube 120 in such a way as to be movable back and forth, a drive shaft 140, which has a distal end provided with a transducer unit 145 which transmits and receives signals and is provided inside the sheath 110 in a rotatable manner, a rigidity changing part 10, which is provided inside the sheath 110, a unit connector 150, which is provided at the proximal side of the outer tube 120 and accommodates the inner shaft 130, a hub 160, which is provided at the proximal side of the inner shaft 130, and a relay connector 170, which interconnects the sheath 110 and the outer tube 120. The diagnostic imaging catheter 100 according to the first embodiment is of a rapid exchange (RX) type in which a guide wire W passes through only the distal portion of the diagnostic imaging catheter 100.

In the context of the present specification, a side of the diagnostic imaging catheter 100 which is inserted into the body lumen is referred to as a distal end or a distal side, a side close to the side of the hub 160 provided in the diagnostic imaging catheter 100 is referred to as a proximal end or a proximal side, and an extending direction of the sheath 110 is referred to as an axial direction.

As illustrated in FIG. 2(A), the drive shaft 140 is positioned in the lumen in the sheath 110 and passes through the sheath 110, the outer tube 120, which is connected to the proximal end of the sheath 110, and the inner shaft 130, which is inserted into the outer tube 120, and extends to the inside of the hub 160. Moreover, the rigidity changing part 10 is located at the internal side of the drive shaft 140 in the radial direction (i.e., the rigidity changing part 10 is radially inward of the drive shaft 140 in this embodiment).

The hub 160, the inner shaft 130, the drive shaft 140, the transducer unit 145, and the rigidity changing part 10 are connected to one another in such a way as to integrally move backward and forward along the axial direction. Therefore, for example, when an operation for the hub 160 to be pushed toward the distal side or in the distal direction is performed, the inner shaft 130, which is connected to the hub 160, is pushed into the outer tube 120 and the unit connector 150, so that the drive shaft 140, the transducer unit 145, and the rigidity changing part 10 move toward the distal side or in the distal direction inside the sheath 110. For example, when an operation for the hub 160 to be pulled toward the proximal side is performed, the inner shaft 130 is pulled out of the outer tube 120 and the unit connector 150 as indicated by arrow “a1” in FIG. 1 and FIG. 2(B), so that the drive shaft 140, the transducer unit 145, and the rigidity changing part 10 move toward the proximal side or in the proximal direction inside the sheath 110 as indicated by arrow “a2”.

As illustrated in FIG. 2(A), when the inner shaft 130 has been most pushed in toward the distal side or in the distal direction, the distal portion of the inner shaft 130 arrives at the vicinity of the relay connector 170. At this time, the transducer unit 145 is situated at the vicinity of the distal end of the sheath 110.

As illustrated in FIG. 2(B), a connector 131 for coming-off prevention is provided at the distal end of the inner shaft 130. The connector 131 for coming-off prevention has a function to prevent the inner shaft 130 from coming off from the outer tube 120. The connector 131 for coming-off prevention is configured to get stuck with a predetermined position of the inner wall of the unit connector 150 when the hub 160 is most pulled toward the proximal side or in the proximal direction, i.e., when the inner shaft 130 is most pulled out from the outer tube 120 and the unit connector 150.

As illustrated in FIG. 3 and FIGS. 4((A) to 4(C), the drive shaft 140 includes a pipe body or tubular body 140a having flexibility, and electric signal cables 140b, which are inserted into or positioned in the pipe body 140a. The flexible tubular body 140a can be comprised of, for example, multiple layers of coils having different winding directions around the axis. Examples of the material of the coils include stainless steel and a nickel-titanium (Ni—Ti) alloy.

As illustrated in FIG. 3 and FIGS. 4((A) to 4(C), the electric signal cables 140b are provided at respective positions opposite in the circumferential direction in a paired manner. In the present embodiment, the electric signal cable 140b is formed in an approximate circular shape in cross-section. The electric signal cables 140b can be configured with, for example, a twisted pair cable or a coaxial cable.

The transducer unit 145 includes an ultrasound transducer (ultrasonic transducer) 145a (corresponding to a signal transmitting and receiving unit), which transmits and receives ultrasound waves (ultrasonic waves), and a housing 145b, which accommodates the ultrasound transducer 145a.

The ultrasound transducer 145a has the function of transmitting ultrasound waves, which serve as inspection waves, into the body lumen, and receiving ultrasound waves reflected from the body lumen. The ultrasound transducer 145a is electrically connected to an electrode terminal 166 (see FIG. 5) via the electric signal cables 140b.

The ultrasound transducer 145a can be formed from a piezoelectric material, such as ceramic or a crystal.

The rigidity changing part 10 makes the rigidity of the drive shaft 140 higher at the proximal side (proximal end) of the drive shaft 140 than at the distal side (or distal end) thereof. As illustrated in FIG. 1, the rigidity changing part 10 is provided in such a way as to extend in the axial direction inside the sheath 110.

As illustrated in FIG. 3, the rigidity changing part 10 is provided in such a way as to extend toward the proximal side or in the proximal direction from a position which is a predetermined distance L1 away from a distal end 140c of the drive shaft 140 toward the proximal side. In other words, at the distal side (distal-most end portion) of the drive shaft 140, there is a region in which the rigidity changing part 10 is not provided (i.e., the distal-most end portion of the drive shaft is devoid of the rigidity changing part 10). Thus, in the distal portion of the drive shaft 140, there is no axial overlap between the drive shaft and the rigidity changing part 10. The rigidity changing part 10 extends up to the inside of a connection pipe 164b of the hub 160 (see FIG. 5).

The above-mentioned distance L1 corresponds to a distance from the distal end 140c of the drive shaft 140 to a distal end 10a of the rigidity changing part 10. The distance L1 is not specifically limited, but is, for example, 150 mm to 200 mm.

As illustrated in FIG. 3 and FIG. 4(A) to 4(C), the rigidity changing part 10 is located at the inner side in the radial direction with respect to the electric signal cables 140b. That is, the rigidity changing part 10 is radially inward of the electric signal cables 140b. The rigidity changing part 10 includes a tapered portion 11, which is provided at the distal end, and a shaft portion 12, which is provided closer to the proximal side than the tapered portion 11.

The tapered portion 11 has a taper shape toward the distal end (the taper portion tapers in the distal direction from a larger outer dimension/diameter to a smaller outer diameter/dimension). The length of the tapered portion 11 in the axial direction is not specifically limited, but is, for example, 150 mm to 200 mm.

The shaft portion 12 is continuous to the proximal end of the tapered portion 11. The shaft portion 12 is configured to extend up to the inside of the connecting pipe 164b in such a way as to have an outer diameter thereof approximately constant.

The material from which the rigidity changing part 10 is made is not specifically limited, but can be metal, such as stainless steel (SUS) or NiTi. For example, in a case where the rigidity changing part 10 is made from SUS, the rigidity of the drive shaft 140 can be advantageously increased. Furthermore, in a case where the rigidity changing part 10 is made from NiTi, since the rigidity changing part 10 has a shape-memory property, the rigidity changing part 10, even when deformed, returns to its original shape. Therefore, when the drive shaft 140 rotates during use of the diagnostic imaging catheter 100, rotational unevenness can be advantageously prevented from occurring in the drive shaft 140.

Since the rigidity changing part 10 is provided in the above-described way, a flexible portion 141 with a relatively low rigidity, an intermediate portion 142 with a rigidity gradually decreasing from the proximal side (proximal end) toward the distal side (distal end), and a high-rigidity portion 143 with a relatively high rigidity are formed in the drive shaft 140 in that order from the distal side (distal end).

The flexible portion 141 corresponds to a region in which the rigidity changing part 10 is not provided in the drive shaft 140. The intermediate portion 142 corresponds to a region in which the tapered portion 11 of the rigidity changing part 10 is provided in the drive shaft 140. The relatively high-rigidity portion 143 corresponds to a region in which the shaft portion 12 of the rigidity changing part 10 is provided in the drive shaft 140.

In this way, since the flexible portion 141, the intermediate portion 142, and the high-rigidity portion 143 are formed in order from the distal side (distal end) in the drive shaft 140, the rigidity of the drive shaft 140 can be made higher at the proximal side (proximal end) than at the distal side (proximal end). Furthermore, since the intermediate portion 142 is formed or located between the flexible portion 141 and the relatively high-rigidity portion 143, the rigidity of the drive shaft 140 can be gradually decreased from the proximal side (proximal end) toward the distal side (distal end) in the intermediate portion 142, so that operability and bending endurance can be improved. Furthermore, in the intermediate portion 142, the outer diameter of the tapered portion 11 gradually decreases in a continuous manner but can be configured to gradually decrease in a stepwise manner.

The sheath 110 is provided in such a way as to extend in the axial direction. As illustrated in FIG. 3, the sheath 110 has a lumen 110a, into which the drive shaft 140 is inserted or positioned in such a way as to be movable back and forth. A guide wire insertion member 114, which is equipped with a guide wire lumen 114a into which a guide wire W is able to be inserted through, is attached to the distal portion of the sheath 110 in such a way as to be arranged in parallel with the lumen 110a provided in the sheath 110. The sheath 110 and the guide wire insertion member 114 can be configured in an integrated fashion with the use of, for example, heat-welding. The guide wire insertion member 114 is provided with a marker 115 having a radiopaque property. The marker 115 is configured to include a metal coil having a high radiopaque property, such as Pt, Au, or Ir.

A communication hole 116, through which the inside and the outside of the lumen 110a communicate with each other, is formed at the distal portion of the sheath 110. Furthermore, a reinforcement member 117, which is configured to strongly bond and support the guide wire insertion member 114, is provided at the distal portion of the sheath 110. A communication path 117a, through which the inside of the lumen 110a located closer to the proximal side than the reinforcement member 117 communicates with the communication hole 116, is formed in the reinforcement member 117. The distal portion of the sheath 110 does not need to be provided with the reinforcement member 117.

The communication hole 116 is a priming liquid discharge hole through which to discharge a priming liquid. When the diagnostic imaging catheter 100 is used, a priming process is performed to fill the inside of the sheath 110 with the priming liquid so as to reduce the attenuation of ultrasound waves caused by air inside the sheath 110 and to efficiently transmit and receive ultrasound waves. When the priming process is performed, the priming liquid is discharged from the communication hole 116 to the outside, and a gas such as air can be discharged together with the priming liquid from the inside of the sheath 110.

The sheath 110 is formed of a material having a high ultrasound transmissivity. The distal portion of the sheath 110, which is the range of area where the ultrasound transducer 145a is moved in the axial direction of the sheath 110, configures an acoustic window portion having ultrasound transmissivity higher than those of other portions.

Each of the sheath 110, the guide wire insertion member 114, and the reinforcement member 117 is formed of a material having flexibility. Such material is not limited to a specific material and includes, for example, various thermoplastic elastomers, such as a styrene elastomer, a polyolefin elastomer, a polyurethane elastomer, a polyester elastomer, a polyamide elastomer, a polyimide elastomer, a polybutadiene elastomer, a trans-polyisoprene elastomer, a fluororubber elastomer, and a chlorinated polyethylene elastomer, and a combination of one or two or more (polymer alloy, polymer blend, or laminated body) of these elastomers can also be used as the material. Furthermore, a hydrophilic lubricant coating layer which exhibits lubricating ability at the time of wetting can be arranged on the outer surface of the sheath 110.

As illustrated in FIG. 5, the hub 160 includes a hub main body 161, which has a hollow structure, a port 162, which communicates with the inside of the hub main body 161, projections 163a and 163b, which are used for direction confirmation to confirm the direction of the hub 160 at the time of connection to the external apparatus 300, a seal member 164a, which seals a portion closer to the proximal side than the port 162, the connection pipe 164b, which holds the drive shaft 140, a bearing 164c, which rotatably supports the connection pipe 164b, and a connector portion 165, inside which an electrode terminal 166 which is configured to be mechanically and electrically connected to the external apparatus 300 is located.

The inner shaft 130 is connected to the distal portion of the hub main body 161. The drive shaft 140 is pulled out from the inner shaft 130 inside the hub main body 161. A protective tube 133 is located between the inner shaft 130 and the drive shaft 140. The protective tube 133 has a function to prevent the occurrence of breakage of the drive shaft 140 due to interference between the inner shaft 130 and the drive shaft 140.

The connection pipe 164b supports the drive shaft 140 and the rigidity changing part 10 at the distal end of the connection pipe 164b, which is an end portion opposite to a rotor 167, so as to transmit rotation of the rotor 167 to the drive shaft 140. The rigidity changing part 10 is fixed to the connection pipe 164b in the vicinity of the proximal end of the rigidity changing part 10. The method for fixing the rigidity changing part 10 and the connection pipe 164b is not specifically limited, but includes, for example, fixation by swaging and bonding by adhesive.

The electric signal cables 140b (see FIG. 3) are inserted through into or positioned in the connection pipe 164b, and one end of each of the electric signal cables 140b is connected to the electrode terminal 166 and the other end of the electric signal cables 140b passes through the drive shaft 140 and is then connected to the ultrasound transducer 145a. A received signal obtained at the ultrasound transducer 145a is transmitted to the external apparatus 300 via the electrode terminal 166 and is then subjected to predetermined processing to be displayed as an image.

Referring back to FIG. 1, the diagnostic imaging catheter 100 is driven in a state of being connected to the external apparatus 300.

As mentioned above, the external apparatus 300 is connected to the connector portion 165 (see FIG. 5) provided at the proximal side of the hub 160.

Furthermore, the external apparatus 300 includes a motor 300a, which is a power source to rotate the drive shaft 140, and a motor 300b, which is a power source to move the drive shaft 140 along the axial direction. The rotational motion of the motor 300b is converted into a motion along the axial direction by a ball screw 300c connected to the motor 300b.

The operation of the external apparatus 300 is controlled by a control apparatus 320, which is electrically connected to the external apparatus 300. The control apparatus 320 includes a central processing unit (CPU) and memory as main constituent components. The control apparatus 320 is electrically connected to a monitor 330.

Next, a usage example or an example of a manner of operation of the diagnostic imaging catheter 100 according to the first embodiment is described.

First, in a state in which the hub 160 is most pulled toward the proximal side or in the proximal direction (see FIG. 2(B)), the operator connects a syringe S filled with a priming liquid to the port 162 and then pushes the plunger of the syringe S to inject the priming liquid into the lumen 110a of the sheath 110.

When the priming liquid is injected into the lumen 110a, the priming liquid is discharged to the outside of the sheath 110 via the communication hole 116, so that a gas such as air can be discharged together with the priming liquid from the inside of the sheath 110 to the outside thereof (a priming process).

After the priming process, the operator connects the external apparatus 300 to the connector portion 165 (see FIG. 5) of the diagnostic imaging catheter 100 as illustrated in FIG. 1. Then, the operator pushes the hub 160 inward until the hub 160 abuts on the proximal end of the unit connector 150 (see FIG. 2(A)) to cause the transducer unit 145 to move to the distal side as illustrated in FIG. 3. In this condition, while the guide wire W is inserted through into the guide wire lumen 114a, the sheath 110 is inserted along the guide wire W to an intended position in the body lumen (for example, a blood vessel). Herein, the diagnostic imaging catheter 100 according to the present embodiment includes the rigidity changing part 10 and, therefore, has flexibility at the distal side and relatively high rigidity at the proximal side. Therefore, operability for the operator is improved.

To acquire a tomographic image at the intended position in the body lumen, the transducer unit 145 moves toward the proximal side or in the proximal direction while rotating together with the drive shaft 140 (a pull-back operation). At this time, the ultrasound transducer 145a of the transducer unit 145 transmits and receives ultrasound waves.

The rotation and movement operations of the drive shaft 140 are controlled by the control apparatus 320. The connector portion 165 provided in the hub 160 is rotated while being connected to the external apparatus 300, and the drive shaft 140 rotates in conjunction with the rotation of the connector portion 165. The rotational speed of the connector portion 165 and the drive shaft 140 is, for example, 1,800 rpm (revolutions per minute).

The ultrasound transducer 145a transmits ultrasound waves into the body based on a signal sent from the control apparatus 320. A signal corresponding to reflected waves received by the ultrasound transducer 145a is sent to the control apparatus 320 via the drive shaft 140 and the external apparatus 300. The control apparatus 320 generates a tomographic image of the body lumen based on a signal sent from the ultrasound transducer 145a, and displays the generated image on the monitor 330.

As described above, the diagnostic imaging catheter 100 according to the present embodiment includes the rotatable drive shaft 140 having a distal end provided with the ultrasound transducer 145a, the sheath 110 into which the drive shaft 140 is inserted or positioned and which extends in an axial direction, and the rigidity changing part 10 provided inside the sheath 110 and configured to result in the rigidity of the drive shaft 140 being higher at a proximal side (proximal portion) than at a distal side (distal portion). According to the diagnostic imaging catheter 100 configured in this way, the rigidity changing part 10 can make the rigidity of the drive shaft 140 higher at the proximal side than at the distal side (i.e., the proximal portion is more rigid than the distal portion). Furthermore, the rigidity changing part 10 is located inside the sheath 110 and is, therefore, able to prevent an increase in outer diameter of the sheath 110. That is, the diagnostic imaging catheter exhibits rigidity characteristics like those described above, yet the outer diameter of the sheath 110 is not increased. Accordingly, the diagnostic imaging catheter 100 which has flexibility at the distal side (distal portion) and has relatively high rigidity at the proximal side (distal portion) without requiring an increase in outer diameter of the sheath 110 can be provided.

Furthermore, the rigidity changing part 10 is located in such a way as to extend toward the proximal side or in the proximal direction from a position which is a predetermined distance L1 away from the distal end 140c of the drive shaft 140 toward the proximal side. According to the diagnostic imaging catheter 100 configured in this way, a region in which the rigidity changing part 10 is not provided in the drive shaft 140 constitutes the flexible portion 141, and a region in which the rigidity changing part 10 is provided in the drive shaft 140 constitutes the high-rigidity portion 143. Therefore, with a relatively simple configuration, the diagnostic imaging catheter 100 having flexibility at the distal side and having relatively high rigidity at the proximal side can be provided.

Furthermore, the rigidity changing part 10 is provided in such a way as to extend in the axial direction. According to the diagnostic imaging catheter 100 configured in this way, the rigidity of the drive shaft 140 can be continuously increased along the axial direction.

Moreover, the drive shaft 140 includes the pipe body 140a having flexibility, and the electric signal cables 140b, which are inserted through into the pipe body 140a, and the rigidity changing part 10 is located at the inner side in the radial direction (radially inward) with respect to the electric signal cables 140b. According to the diagnostic imaging catheter 100 configured in this way, since the rigidity changing part 10 is located inside the drive shaft 140, an increase in outer diameter of the sheath 110 can be more advantageously prevented.

Furthermore, the rigidity changing part 10 includes the tapered portion 11, which is provided at the distal end and possesses a taper shape. According to the diagnostic imaging catheter 100 configured in this way, the intermediate portion 142 is formed between the flexible portion 141 and the high-rigidity portion 143. Therefore, the rigidity of the drive shaft 140 can be gradually decreased in a continuous manner from the proximal side toward the distal side in the intermediate portion 142, so that operability and bending endurance can be improved.

### Modification Example of First Embodiment

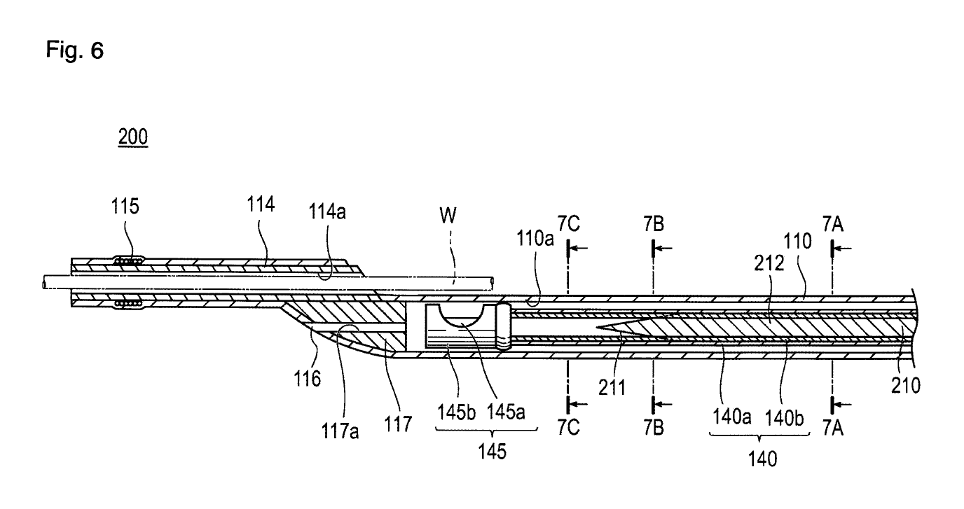

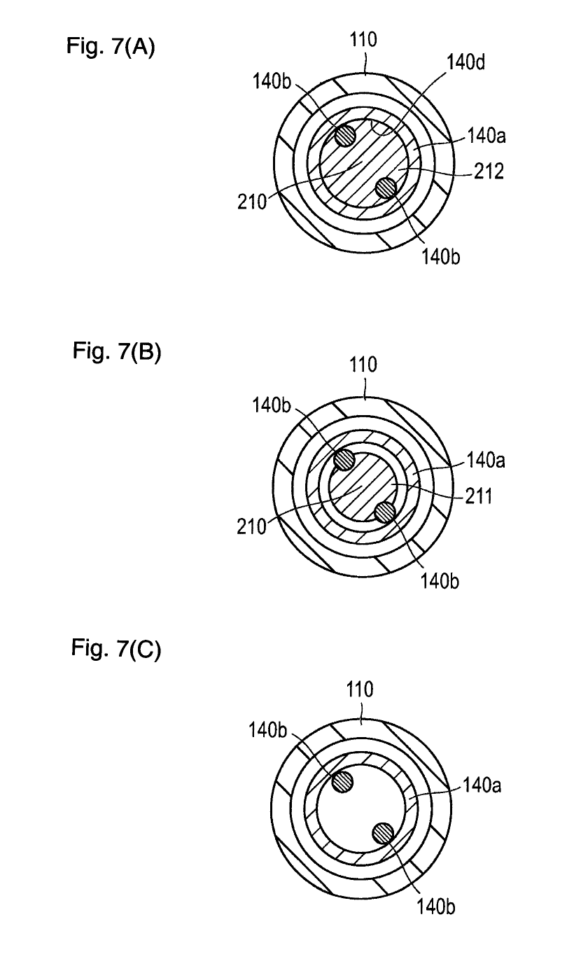

Next, a configuration of a diagnostic imaging catheter 200 according to a modification example of the first embodiment is described with reference to FIG. 6 and FIGS. 7(A), 7(B), and 7(C).

FIG. 6 is an enlarged cross-sectional view illustrating a configuration of the distal side of the diagnostic imaging catheter 200 according to the modification example of the first embodiment, FIG. 7(A) is a cross-sectional view taken along the section line 7A-7A in FIG. 6, FIG. 7(B) is a cross-sectional view taken along the section line 7B-7B in FIG. 6, and FIG. 7(C) is a cross-sectional view taken along the section line 7C-7C in FIG. 6.

The rigidity changing part 10 of the diagnostic imaging catheter 100 according to the first embodiment is located at the inner side in the radial direction (radially inward) with respect to the electric signal cables 140b, as illustrated in FIGS. 4(A) to 4(C). On the other hand, a rigidity changing part 210 of the diagnostic imaging catheter 200 according to the modification example of the first embodiment is located in such a manner that the electric signal cables 140b are buried in or embedded in the rigidity changing part 210, as illustrated in FIG. 6 and FIGS. 7(A) to 7(C). Thus, as shown for example in FIG. 7(A), the electric signal cables 140b are positioned at least partially within the outer confines of the rigidity changing part 210 so that at least some of the transverse cross-section of each of the electric signal cables 140b is positioned radially inwardly of the outer periphery of the rigidity changing part 210.

The rigidity changing part 210 includes a tapered portion 211, which is provided at the distal end, and a shaft portion 212, which is provided closer to the proximal side (proximal end) than the tapered portion 211, as illustrated in FIG. 6 and FIGS. 7(A) to 7(C).

The shaft portion 212 extends up to the inside of the connecting pipe 164b and possesses an outer diameter that is approximately constant. The electric signal cables 140b are buried in the shaft portion 212, as illustrated in FIGS. 7(A) to 7(C). Furthermore, the shaft portion 212 is located in such a way as to infill or fill-up a lumen 140d of the pipe body 140a. In other words, the outer diameter of the shaft portion 212 is approximately the same as the diameter (inner diameter) of the lumen 140d of the pipe body 140a so that the shaft portion 212 fills the lumen in the tubular body 140a considered with reference to a transverse cross-section such as shown in FIG. 7(A).

The material used to configure the rigidity changing part 210 is not specifically limited as long as the electric signal cable 140b can be buried in the rigidity changing part 210. Examples of materials include a resin such as epoxy.

As described above, in the diagnostic imaging catheter 200 according to the modification example of the first embodiment, the rigidity changing part 210 includes the shaft portion 212, in which the electric signal cables 140b are buried and which is located in such a way as to infill or fill-up the lumen 140d of the pipe body or tubular body 140a. According to the diagnostic imaging catheter 200 configured in this way, since the shaft portion 212 of the rigidity changing part 210 is located in such a way as to infill or fill-up the lumen 140d of the pipe body 140a, the shaft portion 212 can be configured to be thicker (great outer diameter) than the shaft portion 12 in the first embodiment. Accordingly, the rigidity of the drive shaft 140 in the high-rigidity portion 143 is greater than in the diagnostic imaging catheter 100 according to the first embodiment. Furthermore, since the shaft portion 212 in which the electric signal cables 140b are buried can be relatively easily inserted into the lumen 140d of the pipe body 140a, the diagnostic imaging catheter 200 can be relatively easily manufactured.

### Second Embodiment

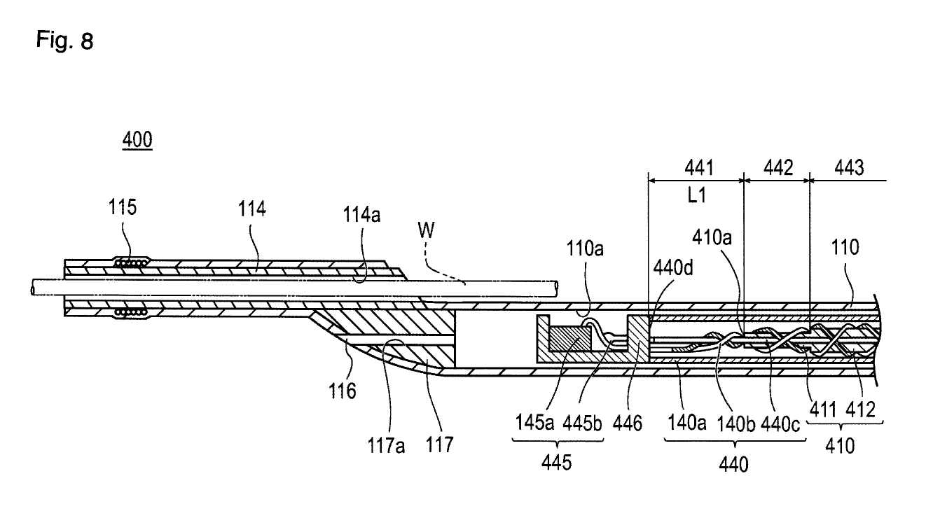

Next, a configuration of a diagnostic imaging catheter 400 according to a second embodiment is described with reference to FIG. 8.

FIG. 8 is an enlarged cross-sectional view illustrating a configuration of the distal side of the diagnostic imaging catheter 400 according to the second embodiment. Features in the second embodiment that are common to or the same as those in the first embodiment are identified by a common reference numeral and a detailed description of such features is not repeated. The description which follows primarily discusses aspects of this second embodiment differing from the embodiment and modification described above. The second embodiment differs from the first embodiment in terms of the configuration of a drive shaft 440, a signal transmitting and receiving unit 445, and a rigidity changing part 410.

The diagnostic imaging catheter 400 according to the second embodiment is of a dual type which has both the functions of IVUS and optical coherence tomography (OCT) and is configured to switch between the two functions and to operate concurrently using both functions.

As illustrated in FIG. 8, the diagnostic imaging catheter 400 according to the second embodiment includes the drive shaft 440, which has a distal end equipped with the signal transmitting and receiving unit 445, which transmits and receives signals, and which is rotatably provided in the sheath 110, and the rigidity changing part 410, which is provided inside the sheath 110. The configuration of the sheath 110, the outer tube 120, the inner shaft 130, the unit connector 150, the hub 160, and the relay connector 170 is similar to the configuration of those features in the diagnostic imaging catheter 100 described in the first embodiment, and so a detailed description of such aspects is not repeated.

As illustrated in FIG. 8, the drive shaft 440 includes a pipe body 140a, electric signal cables 140b, and an optical fiber cable 440c, which is inserted through into or is positioned in the pipe body 140a.

The signal transmitting and receiving unit 445 includes an ultrasound transducer 145a and an optical transceiver 445b, which transmits and receives light. The ultrasound transducer 145a and the optical transceiver 445b are accommodated in a housing 446.

The optical transceiver 445b continuously transmits the transferred measurement light to the inside of the body lumen and continuously receives reflected light from a biological tissue in the body lumen. The optical transceiver 445b is provided at the distal end of the optical fiber cable 440c and includes a ball lens (optical element) which has a lens function of condensing light and a reflection function of reflecting light.

The proximal end of the housing 446 is connected to the drive shaft 440.

By virtue of the rigidity changing part 410, the rigidity of the drive shaft 440 is higher at the proximal side (proximal end) of the drive shaft 440 than at the distal side (distal end) of the drive shaft 440. The rigidity changing part 410 is provided inside the sheath 110 and extends in the axial direction.

As illustrated in FIG. 8, the rigidity changing part 410 extends toward the proximal side or in the proximal direction from a position which is a predetermined distance L1 away from a distal end 440d of the drive shaft 440 toward the proximal side. The rigidity changing part 410 covers or surrounds (encircles) the outer periphery of the optical fiber cable 440c in such a manner that the distal side (distal portion) of the optical fiber cable 440c is exposed only as much as the distance L1. The rigidity changing part 410 is bonded to the optical fiber cable 440c by, for example, adhesive.

As illustrated in FIG. 8, the rigidity changing part 410 includes a small-diameter portion 411, which is provided at the distal side, and a large-diameter portion 412, which is provided at the proximal side of the small-diameter portion 411 and the diameter (outer diameter) of which is larger than that of the small-diameter portion 411.

The above-mentioned distance L1 corresponds to a distance from the distal end 440d of the drive shaft 440 to the distal end 410a of the rigidity changing part 410.

As illustrated in FIG. 8, the rigidity changing part 410 is located on the outer periphery of the optical fiber cable 440c and at the inner side in the radial direction (radially inward) with respect to the electric signal cables 140b.

The material used to configure the rigidity changing part 410 is not specifically limited as long as it is able to cover or surround (encircle) the optical fiber cable 440c, but can be, for example, polyimide or polycarbonate.

Since the rigidity changing part 410 is provided in the above-described way, a flexible portion 441 with a relatively low rigidity, an intermediate portion 442 with a rigidity higher than that of the flexible portion 441, and a relatively high-rigidity portion 443 with a rigidity higher than that of the intermediate portion 442 are formed in the drive shaft 440 in that order from the distal side (distal end).

The flexible portion 441 corresponds to a region in which the rigidity changing part 410 is not provided in the drive shaft 440 (i.e., the drive shaft 440 is devoid of the rigidity changing part 410). The intermediate portion 442 corresponds to a region in which the small-diameter portion 411 is provided in the drive shaft 440. The relatively high-rigidity portion 443 corresponds to a region in which the large-diameter portion 412 is provided in the drive shaft 440.

In this way, since the flexible portion 441, the intermediate portion 442, and the high-rigidity portion 443 are formed in that order from the distal side (distal end) in the drive shaft 440, the rigidity of the drive shaft 440 can be made higher at the proximal side than at the distal side.

As described above, in the diagnostic imaging catheter 400 according to the second embodiment, the drive shaft 440 includes the pipe body 140a, which has flexibility, and the optical fiber cable 440c, which is inserted through into the pipe body 140a, and the rigidity changing part 410 is located in such a way as to coat the outer periphery of the optical fiber cable 440c. According to the diagnostic imaging catheter 400 configured in this way, a diagnostic imaging catheter 400 having flexibility at the distal side and having high rigidity at the proximal side can be provided.

### Third Embodiment

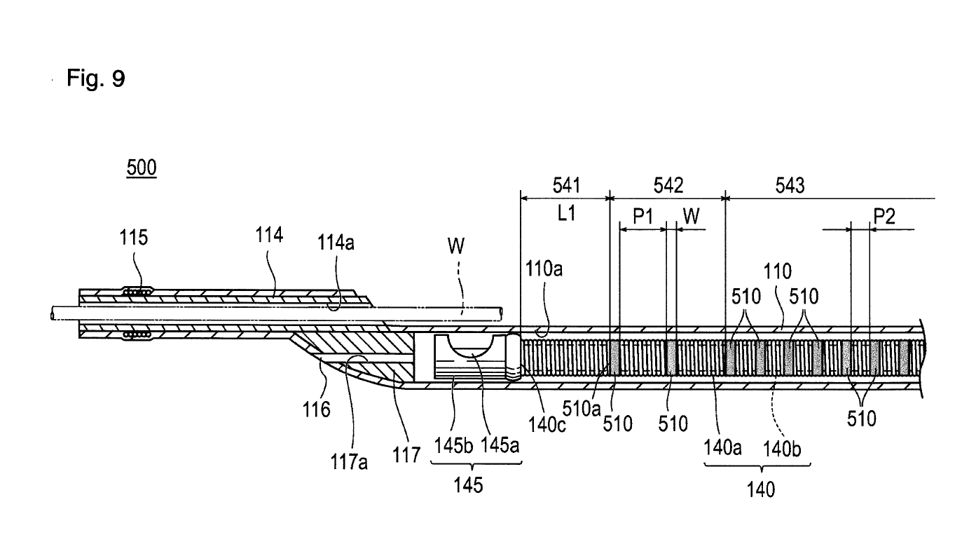

Next, a configuration of a diagnostic imaging catheter 500 according to a third embodiment is described with reference to FIG. 9.

FIG. 9 is an enlarged cross-sectional view illustrating a configuration of the distal side of the diagnostic imaging catheter 500 according to the third embodiment. Features in the third embodiment that are common to or the same as those in the first embodiment are identified by a common reference numeral and a detailed description of such features is not repeated. The description which follows primarily discusses aspects of this second embodiment differing from the embodiment and modification described above. The third embodiment differs from the first embodiment in terms of the configuration of a rigidity changing part 510.

The diagnostic imaging catheter 500 according to the third embodiment is applied to IVUS as with the diagnostic imaging catheter 100 in the first embodiment.

As illustrated in FIG. 9, the diagnostic imaging catheter 500 according to the third embodiment includes the rigidity changing part 510, which is provided inside the sheath 110. The configuration of the sheath 110, the outer tube 120, the inner shaft 130, the drive shaft 140, the unit connector 150, the hub 160, and the relay connector 170 is similar to those same features in the diagnostic imaging catheter 100 described in the first embodiment and so a detailed description of such features is not repeated.

The rigidity changing part 510 results in the rigidity of the drive shaft 140 being higher at the proximal side (proximal portion) of the drive shaft 140 than at the distal side (distal portion) of the drive shaft 140. As illustrated in FIG. 9, the rigidity changing part 510 is intermittently arranged toward the proximal side or in the proximal direction from a position which is a predetermined distance L1 away from the distal end 140c of the drive shaft 140 toward the proximal side. The rigidity changing part 510 extends to the proximal end of the drive shaft 140.

The above-mentioned distance L1 corresponds to a distance from the distal end 140c of the drive shaft 140 to the distal end 510a of the rigidity changing part 510.

As illustrated in FIG. 9, the rigidity changing part 510 is intermittently arranged in such a manner that the rigidity changing part 510 is more densely arranged at the proximal side (proximal portion) of the drive shaft 140 than at the distal side (distal portion) of the drive shaft 140. The rigidity changing part 510 is formed by performing brazing, such as soldering, on the outer surface of the tubular body 140a. The rigidity changing part 510 can be formed on one round of the outer periphery of the tubular body 140a, or can be formed on at least a part thereof in the circumferential direction. That is, the rigidity changing part 510 can be configured to have either a circumferential extent of 360° so that the rigidity changing part 510 encircles the entire circumferential extent of the tubular body 140a or a circumferential extent of less than 360° so that the rigidity changing part 510 encircles a partial circumferential extent of the tubular body 140a.

Furthermore, the rigidity changing part 510 can be formed by swaging a ring or annular member made of metal (platinum or SUS) or a short pipe to the tubular body 140a at the intermittent or spaced apart locations shown in FIG. 9.

As illustrated in FIG. 9, the rigidity changing part 510 is arranged at a first pitch P1 at the distal side and is arranged at a second pitch P2 at the proximal side. The first pitch P1 is larger than the second pitch P2. The first pitch P1 is not specifically limited, but is, for example, 10 mm to 20 mm. Furthermore, the second pitch P2 is not specifically limited, but is, for example, 5 mm to 7 mm. Moreover, the width W of each section or part of the rigidity changing part 510 is not specifically limited, but is, for example, 0.5 mm to 1 mm.

Since the rigidity changing part 510 is provided in the above-described way, a flexible portion 541 with a relatively low rigidity, an intermediate portion 542 with a rigidity higher than that of the flexible portion 541, and a high-rigidity portion 543 with a rigidity higher than that of the intermediate portion 542 are formed in the drive shaft 140 in order from the distal side.

The flexible portion 541 corresponds to a region in which the rigidity changing part 510 is not provided in the drive shaft 140. The intermediate portion 542 corresponds to a region in which the rigidity changing part 510 is arranged at the first pitch P1 in the drive shaft 140. The high-rigidity portion 543 corresponds to a region in which the rigidity changing part 510 is arranged at the second pitch P2 in the drive shaft 140.

In this way, since the flexible portion 541, the intermediate portion 542, and the high-rigidity portion 543 are formed in that order from the distal side (distal end) in the drive shaft 140, the rigidity of the drive shaft 140 can be made higher at the proximal side than at the distal side.

As described above, in the diagnostic imaging catheter 500 according to the third embodiment, the rigidity changing part 510 is intermittently provided in the axial direction in such a manner that the rigidity changing part 510 is more densely arranged at the proximal side (proximal portion) than at the distal side (distal portion). According to the diagnostic imaging catheter 500 configured in this way, a diagnostic imaging catheter 500 having flexibility at the distal side (distal portion) and relatively high rigidity at the proximal side (proximal portion) can be provided. Furthermore, the rigidity changing parts 510 can be reduced in weight as compared with a configuration in which a rigidity changing part is continuously provided.

While the inventive diagnostic imaging catheters disclosed here have been described above by way of the embodiments and a modification example representing examples of the inventive diagnostic imaging catheter, the invention is not limited to the configurations described in the embodiments and modification example, but can be modified or altered as appropriate based on the description of claims.

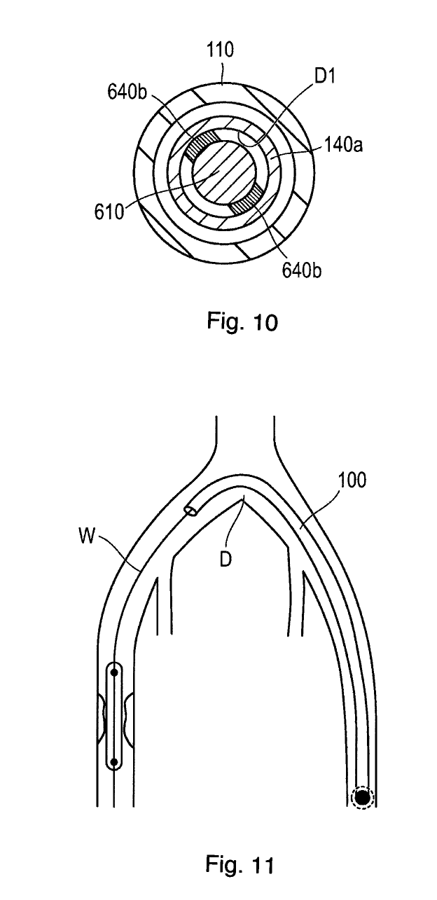

For example, in the above-described first embodiment, the electric signal cable 140b possesses an approximately circular shape in transverse cross-section as illustrated in FIGS. 4(A) to 4(C). However, as illustrated in FIG. 10, electric signal cables 640b can be configured to be located in a clearance D1 between the tubular body 140a and the rigidity changing part 610. In this embodiment, the transverse cross-sectional shape of the electric signal cables 640b is not circular, but rather is a cross-section that matches the cross-section of a limited circumferential extent of the space between the tubular body 140a and the rigidity changing part 610. Configuring the electric signal cables 640b in this way makes it possible to make the outer diameter of the rigidity changing part 610 larger than the outer diameter of the rigidity changing part 10 of the diagnostic imaging catheter 100 in the first embodiment. Therefore, the rigidity of the drive shaft is increased.

Furthermore, when the diagnostic imaging catheter 100 according to the first embodiment is applied to a case where the disease site is a peripheral site such as a lower extremity, as illustrated in FIG. 11, there is a procedure to cause the diagnostic imaging catheter to approach from a lower extremity opposite to the disease site. At this time, in a bifurcated portion D of the iliac artery illustrated in FIG. 11, the friction between the drive shaft and the sheath increases. Accordingly, it is desirable that, in a position corresponding to the bifurcated portion D in the diagnostic imaging catheter 100, the outer diameter of the rigidity changing part be made smaller to partially decrease the rigidity of the high-rigidity portion 143 in the drive shaft 140.

Furthermore, while, in the above-described first embodiment, the rigidity changing part 10 of the diagnostic imaging catheter 100 includes the tapered portion 11, the tapered portion 11 does not need to be provided.

Moreover, in the above-described first embodiment, as illustrated in FIG. 3, the rigidity changing part 10 is located in such a way as to extend toward the proximal side or in the proximal direction from a position which is a predetermined distance L1 away from the distal end 140c of the drive shaft 140 toward the proximal side. However, the rigidity changing part can be located in such a way as to extend toward the proximal side from the distal end 140c of the drive shaft 140. At this time, the outer diameter of the rigidity changing part is configured to be smaller at the distal end than at the proximal end.

Furthermore, in the above-described second embodiment, the diagnostic imaging catheter 400 is applied to a dual type having both the functions of IVUS and OCT, but can be applied to OCT.

Moreover, in the above-described third embodiment, the diagnostic imaging catheter 500 is applied to IVUS, but can be applied to OCT or a dual type having both the functions of IVUS and OCT.

The detailed description above describes embodiments and a modification of a diagnostic imaging catheter representing examples of the inventive diagnostic imaging catheter disclosed here. The invention is not limited, however, to the precise embodiments and modification described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A diagnostic imaging catheter comprising:
a rotatable drive shaft possessing a distal end at which is located a signal transmitting and receiving unit that transmits signals towards an inner surface of a lumen in a living body and receives reflected signals that are reflected from the inner surface of the lumen in the body;
a sheath in which the drive shaft is positioned and which extends in an axial direction; and
a rigidity changing part that is positioned inside the sheath and that imparts a higher rigidity to a proximal portion of the drive shaft than a distal portion of the drive shaft.

2. The diagnostic imaging catheter according to claim 1, wherein the rigidity changing part possesses a distal end that is proximally spaced from the distal end of the drive shaft by a predetermined distance, the rigidity changing part extending in a proximal direction from the distal end of the rigidity changing part toward a proximal end of the rigidity changing part.

3. The diagnostic imaging catheter according to claim 1, wherein the rigidity changing part extends in the axial direction.

4. The diagnostic imaging catheter according to claim 1, wherein the drive shaft includes:
a flexible tubular body; and
electric signal cables positioned inside the tubular body; and
the rigidity changing part being positioned radially inwardly with respect to the electric signal cables.

5. The diagnostic imaging catheter according to claim 1, wherein the drive shaft includes:
a flexible tubular body; and
electric signal cables positioned inside the tubular body; and
the rigidity changing part including a shaft portion in which the electric signal cables are embedded and which is located in such a way as to infill a lumen of the pipe body.

6. The diagnostic imaging catheter according to claim 5, wherein the tubular body includes a lumen in which the rigidity changing part is located, the shaft part of the rigidity changing part completely filling the lumen of the tubular body.

7. The diagnostic imaging catheter according to claim 1, wherein the drive shaft includes:
a flexible tubular body; and
an optical fiber cable positioned in the tubular body;
the rigidity changing part encircling an outer periphery of the optical fiber cable.

8. The diagnostic imaging catheter according to claim 1, wherein the rigidity changing part includes a distal end portion that tapers in a distal direction from a larger size to a smaller size.

9. The diagnostic imaging catheter according to claim 1, wherein the rigidity changing part is intermittently provided in the axial direction such that the rigidity changing part is more densely arranged at the proximal portion of the rigidity changing part than at the distal portion of the rigidity changing part.

10. A diagnostic imaging catheter comprising:
an axially extending sheath that includes a lumen extending along an axial extent of the sheath;
an axially extending rotatable drive shaft positioned in the lumen of the sheath, the drive shaft being axially movable and possessing a distal end at which is located a signal transmitting and receiving unit that transmits signals towards an inner surface of a lumen in a living body and receives reflected signals that are reflected from the inner surface of the lumen in the living body;
the drive shaft possessing a distal portion terminating in a distal direction at the distal end of the drive shaft, the drive shaft also possessing a proximal portion terminating in a proximal direction at a proximal end of the drive shaft;
a rigidity changing part that is positioned in the lumen in the sheath and that imparts a higher rigidity to a proximal portion of the drive shaft than the distal portion of the drive shaft;
the rigidity changing part being connected to the drive shaft so that axial movement of the drive shaft results in axial movement of the rigidity changing part, the rigidity changing part possessing a distal portion that terminates in a distal direction at the distal end of the rigidity changing part; and
the distal end of the rigidity changing part being axially spaced in the proximal direction from the distal end of the drive shaft so that the distal portion of the drive shaft does not axially overlap the rigidity changing part, the rigidity changing part axially overlapping with the drive shaft.

11. The diagnostic imaging catheter according to claim 10, wherein the drive shaft includes a flexible tubular body and electric signal cables positioned inside the tubular body, the rigidity changing part possessing an outer periphery positioned radially inwardly of the electric signal cables.

12. The diagnostic imaging catheter according to claim 10, wherein the drive shaft includes a flexible tubular body and electric signal cables positioned inside the tubular body, the rigidity changing part including a shaft portion in which the electric signal cables are embedded and which fills a transverse cross-section of the lumen in the tubular body.

13. The diagnostic imaging catheter according to claim 12, wherein the tubular body includes a lumen extending along a length of the tubular body and in which the rigidity changing part is located, the shaft portion of the rigidity changing part fills a transverse cross-section of the lumen in the tubular body.

14. The diagnostic imaging catheter according to claim 10, wherein the drive shaft includes a flexible tubular body and an optical fiber cable positioned in the tubular body, the rigidity changing part encircling an outer periphery of the optical fiber cable.

15. The diagnostic imaging catheter according to claim 10, wherein the rigidity changing part includes a shaft portion and a tapered portion, the tapered portion being located at a distal end of the shaft portion, the tapered portion tapering in the distal direction from a larger outer diameter to a smaller outer diameter, the shaft portion possessing a constant outer diameter extending from the distal end of the shaft portion toward a proximal end of the shaft portion.

16. The diagnostic imaging catheter according to claim 10, wherein the rigidity changing part includes a plurality of rings fixed to the drive shaft at spaced apart locations such that the rings are more densely arranged at the proximal portion of the rigidity changing part than at the distal portion of the rigidity changing part.

17. A method comprising:
introducing a distal end of a diagnostic imaging catheter into a lumen in a living body, the diagnostic imaging catheter comprising:
a rotatable drive shaft possessing a distal end at which is located a signal transmitting and receiving unit;
a sheath in which the drive shaft is positioned and which extends in an axial direction; and
a rigidity changing part that is positioned inside the sheath and that imparts a higher rigidity to a proximal portion of the drive shaft than a distal portion of the drive shaft;
moving the diagnostic imaging catheter in the lumen of the living body to position the signal transmitting and receiving unit at a desired position in the lumen in the living body;
transmitting signals from the signal transmitting and receiving unit towards an inner surface of the lumen in the living body and receiving reflected signals that are reflected from the inner surface of the lumen in the body; and
generating an image of the lumen in the living body using the reflected signals.
